(19) United States
(12) Patent Application Publication
(10) Pub. No.: US 2025/0123231 A1
(43) Pub. Date: Apr. 17, 2025

(54) SOIL MOISTURE SENSOR PROBE

(71) Applicant: METER Group, Inc., Logan, UT (US)

(72) Inventors: Andreas Steins, Sturnberg (DE); Georg von Unold, München (DE); Douglas R. Cobos, Pullman, WA (US); Manuel Irritier, München (DE); Gaylon S. Campbell, Pullman, WA (US)

(21) Appl. No.: 18/755,214

(22) Filed: Jun. 26, 2024

Related U.S. Application Data
(63) Continuation-in-part of application No. 29/914,324, filed on Oct. 16, 2023.
(60) Provisional application No. 63/629,974, filed on Mar. 13, 2024.

(51) Int. Cl. G01N 27/22; G01N 33/24

(52) U.S. Cl. CPC G01N 27/223; G01N 27/221; G01N 33/245; G01N 33/246

(57) ABSTRACT

A dielectric sensor probe includes a stake having a longitudinal axis and an electric component positioned in the stake. The electronic component includes a first electrode having a first length measured along the longitudinal axis and a second electrode having a second length measured along the longitudinal axis, the second electrode being spaced apart from the first electrode. The first length and the second length longitudinally overlap each other. The electronic component is operable to measure dielectric permittivity of a quantity of material positioned between the first electrode and the second electrode.

### CROSS REFERENCE TO RELATED APPLICATIONS

This claims priority to U.S. Provisional Patent Application No. 63/629,974, filed 13 Mar. 2024 and entitled “Soil Moisture Sensor Probe,” and is a continuation-in-part of U.S. Design patent application Ser. No. 29/914,324, filed 16 Oct. 2023 and entitled “Soil Sensor,” the entire disclosures of which are hereby incorporated by reference.

### FIELD

The described embodiments relate generally to soil sensors and more particularly to soil sensors for agriculture.

### BACKGROUND

Soil moisture/water content and soil permittivity levels can be important factors in various fields, such as agricultural production, drought and flood forecasting, forest fire prediction, water supply management, irrigation control, precision agriculture, etc. For example, in agricultural applications, many crops obtain most of their water via the soil. Crops can be unhealthy or die if the levels of soil moisture, fertilizer, and other soil properties are inappropriate. However, typical sensors placed in the soil can provide inconsistent readings due to inconsistencies in the soil media such as rocks, voids or air gaps between the sensor and the soil, excessive water collection at the surface of the sensor, or other non-homogeneous soil conditions. Furthermore, soil moisture levels can vary depending on the depth and condition of the soil, and soil sensors may be ineffective at measuring soil properties at multiple different depths. There is a constant need for improvements to existing soil sensors.

### SUMMARY

In at least one example, a dielectric sensor probe can include an electronic component positioned in the stake. The electronic component positioned in the stake can include a first electrode having a first length measured along the longitudinal axis and a second electrode having a second length measured along the longitudinal axis. The first length and the second length longitudinally overlap each other. The electronic component is operable to measure dielectric permittivity of a quantity of material positioned between the first electrode and the second electrode.

In one example of the dielectric sensor probe, the stake includes a first fin and a second fin. The first electrode is positioned in the first fin and the second electrode is positioned in the second fin.

In one example of the dielectric sensor probe, the first electrode and the second electrode are angularly offset from each other about the longitudinal axis.

In one example of the dielectric sensor probe, the electronic component includes a first circuit board and a second circuit board arranged in an x-shape. The first electrode is positioned on the first circuit board, and the second electrode is positioned on the second circuit board.

In one example of the dielectric sensor probe, the stake includes a shell, and the electronic component extends through the shell.

In one example of the dielectric sensor probe, the stake includes a tapered bottom end.

In one example of the dielectric sensor probe, the electronic component includes a third electrode having a third length measured along the longitudinal axis. The third electrode is spaced apart from the first electrode. The electronic component is operable to measure dielectric permittivity of a second quantity of material positioned between the first electrode and the third electrode.

In one example of the dielectric sensor probe, the electronic component includes a third electrode having a third length measured along the longitudinal axis. The third electrode is spaced apart from the first electrode in a direction parallel to the longitudinal axis. The electronic component includes a fourth electrode having a fourth length measured along the longitudinal axis. The fourth electrode is longitudinally spaced apart from the second electrode. The third length and the fourth length longitudinally overlap each other. The electronic component is operable to measure dielectric permittivity of a second quantity of material positioned between the third electrode and the fourth electrode.

In one example of the dielectric sensor probe, the dielectric sensor probe further includes a set of rigid members extending through an internal volume defined in the stake and laterally surrounding the electronic component. The rigid members are configured to transfer a load applied to a top end of the sensor probe to a bottom tip of the sensor probe.

In at least one example, a soil sensor includes a shank body including a pointed tip, the shank body having a cross-sectional profile including at least two fins extending radially from a longitudinal axis of the shank body and at least two electrodes coupled with the shank body and operable to measure dielectric permittivity of a quantity of material positioned laterally adjacent to the at least two fins of the shank body.

In one example of the soil sensor, the at least two electrodes include a first electrode positioned in a first fin of the at least two fins and a second electrode positioned in a second fin of the at least two fins.

In one example of the soil sensor, the at least two electrodes overlap a longitudinal position in the shank body.

In one example of the soil sensor, the cross-sectional profile includes at least two additional fins. The at least two fins and the at least two additional fins are arranged in an X-shape.

In one example of the soil sensor, the soil sensor further includes at least two additional electrodes coupled with the shank body in the at least two additional fins and an electronic component in electronic communication with the at least two electrodes and the at least two additional electrodes and configured to sequentially measure dielectric permittivity of the quantity of material and at least one additional quantity of material laterally adjacent to the quantity of material.

In one example of the soil sensor, the cross-sectional profile includes a central substantially cylindrical portion defining an inner cavity.

In at least one example, a method of implementing a dielectric sensor in a porous medium includes forming a bore in a porous medium, the bore having a bore diameter and inserting a dielectric sensor into the porous medium. The dielectric sensor includes a central portion, at least two fins, and at least two electrodes positioned in the at least two fins. Inserting the dielectric sensor into the porous medium includes inserting the central portion of the dielectric sensor into the bore and inserting the at least two fins into the porous medium external to the bore diameter, wherein the porous medium is positioned between the at least two electrodes.

In one example of the method of implementing a dielectric sensor in a porous medium, the method further includes coupling a driving tool to a top end of the dielectric sensor and inserting the dielectric sensor into the porous medium via the driving tool.

In one example of the method of implementing a dielectric sensor in a porous medium, the method further includes measuring permittivity of the porous medium using the at least two electrodes.

In one example of the method of implementing a dielectric sensor in a porous medium, the dielectric sensor includes at least one additional fin and at least one additional electrode positioned in the at least one additional fin.

In one example of the method of implementing a dielectric sensor in a porous medium, the method further includes measuring permittivity of a first quantity of the porous medium using the at least two electrodes and measuring permittivity of a second quantity of the porous medium using one of the at least two electrodes and the at least one additional electrode.

### BRIEF DESCRIPTION OF THE DRAWINGS

FIG. 1 illustrates a perspective view of a dielectric sensor probe.

FIG. 2 illustrates a side view diagram of the dielectric sensor probe of FIG. 1 positioned within the ground.

FIG. 3 illustrates an exploded view of the probe of FIG. 1.

FIG. 4A illustrates a perspective view of the probe of FIG. 1 with the outer shell removed.

FIG. 4B is a detail view of the end of the probe within zone 4B-4B in FIG. 4A.

FIG. 5 illustrates a cross-sectional view of the probe of FIG. 1 illustrating an example electric field emitted from the probe.

FIG. 6 illustrates a side view of a portion of the probe of FIG. 1 and showing the electric field of FIG. 5.

FIG. 7 illustrates a perspective view of the cap, the tapered bottom end, and the set of rigid members of the probe of FIGS. 3-4B.

FIG. 8 illustrates a perspective view of the top end of the probe of FIGS. 3-4A.

FIG. 9 illustrates an auger for forming a bore for installation of the probe.

FIG. 10 illustrates a driving tool for inserting the probe into the bore.

FIG. 11 illustrates a system for collecting data from the probe.

FIG. 12 illustrates a removal lever mechanism for removing the probe from the ground.

FIG. 13 is a flowchart illustrating a method of implementing a dielectric sensor probe in a porous medium.

### DETAILED DESCRIPTION

Before any embodiments of the disclosure are explained in detail, it is to be understood that the embodiments of the disclosure are not limited in their application to the details of construction and the arrangement of components set forth in the following description or illustrated in the associated drawings. The embodiments are capable of other configurations and of being practiced or of being carried out in various ways. Numbers provided in flow charts and processes are provided for clarity in illustrating steps and operations and do not necessarily indicate a particular order or sequence. Unless otherwise defined, the term “or” can refer to a choice of alternatives (e.g., a disjunction operator, or an exclusive or) or a combination of the alternatives (e.g., a conjunction operator, and/or, a logical or, or a Boolean OR). Unless otherwise defined, “connected” can refer to an electrical or mechanical connection. Relative terms such as “about,” “approximately,” or “substantially” indicate that absolute exactness is not required and that features or elements being modified by such terms are within acceptable tolerances as would be recognized by one of ordinary skill in the art. For example, as used herein, the term “substantially perpendicular” shall be interpreted to include any orientation within five degrees of perpendicular, or from between 85 and 95 degrees.

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The following disclosure relates to soil sensor equipment, including probes, stakes, prongs, poles, struts, piles, shafts, similar sensor structures, and combinations thereof and their connected sensors (e.g., electrodes, thermal sensors, resistive sensors, similar sensors, and combinations thereof), which are collectively be referred to as “probes” in the present disclosure to avoid repetition. Soil probes can include dielectric sensor probes for measuring soil moisture levels and other properties. In particular, the following disclosure relates a dielectric sensor probe which, in some embodiments, can measure soil moisture levels at multiple soil depths using multiple pairs of electrodes longitudinally spaced along the portions of the probe inserted into the ground. The dielectric sensor probe can improve measurements of soil moisture levels, even in the presence of obstacles or non-homogenous features in the ground surrounding the probe such as water, voids, sand, mud, stones, or other buried objects.

The dielectric sensor probe can include sets of multiple electrodes configured to all have a common depth when the probe is vertically inserted into the ground, i.e., at a common length position along the inserted portion of the probe. Moisture levels can be measured between each pair of adjacent electrodes of the multiple electrodes at the same depth, so multiple sensor readings can be collected and processed to help reduce error caused by non-homogenous features in the soil at near the electrodes. For example, multiple sensor readings may be analyzed for different portions of soil surrounding the probe to detect abnormalities such as the presence of a rock or an air pocket, and those abnormalities can be accounted for to obtain more accurate sensor readings.

The dielectric sensor probe can include additional sets of multiple electrodes positioned longitudinally along the length of the soil probe. Each set of multiple electrodes can obtain multiple measurements at its corresponding depth within the ground and corresponding to different portions of the soil surrounding the probe.

Ground-inserted soil sensor probes are conventionally installed by inserting the probe into a pre-formed bore in the ground. The bore is practically always conical in shape, with a wider top diameter than its bottom diameter. Conventional probes have a cylindrical probe body inserted into the bore, so at least a small gap initially remains between the cylindrical probe surface and the bore wall surface. Water can be funneled into this gap in a manner that can reduce the accuracy of soil moisture measurements. Sensor probes of the present disclosure can be include a cross-shaped (or x-shaped) cross-section that is insertable into the ground without a pre-formed opening for the outer peripheral tips of the fins of the cross-sectional shape. A cross-, x-, y-, or other finned cross-sectional shape of a probe body can allow the dielectric sensor probe to be installed or bored into the ground while minimizing water funnel towards the bore, thereby improving accuracy. With a multi-fin geometry, soil measured with highest sensitivity may sit in between each pair of fins/antennae. Accordingly, gaps are not as significant of an issue as with cylindrical probes, where the sensing sensitivity drastically decreases with distance to the probe and gaps, which usually appear during the soil drying process, introduce significant errors in collected data.

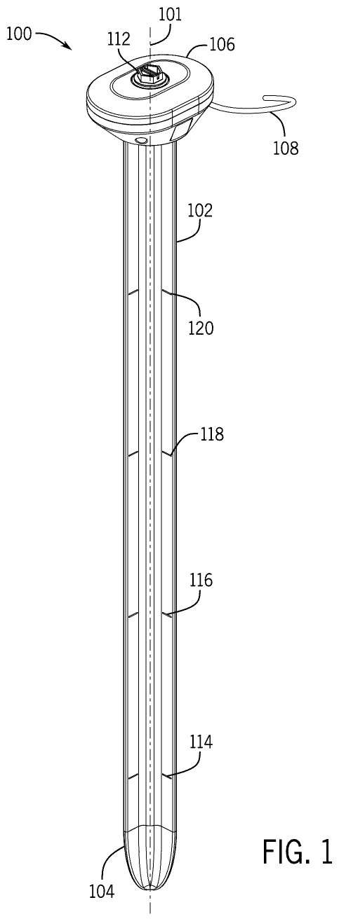

These and other embodiments are discussed below with reference to FIGS. 1-13. However, those skilled in the art will readily appreciate that the detailed description given herein with respect to these figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1 illustrates a perspective view of a dielectric sensor probe 100. The dielectric sensor probe 100 can be a soil sensor configured to measure a dielectric permittivity, and/or moisture/water content of soil. As used herein, dielectric permittivity refers to the ability of a substance or material to hold an electrical charge. For example, a larger dielectric permittivity exhibits larger dielectric polarizability than a material with a lower dielectric permittivity, and thus can store a greater amount of energy.

The dielectric sensor probe 100 can include a stake 102. The stake 102 can be a shank body, pole, post, spike, or pile. The stake 102 can have a central longitudinal axis 101. In particular, the stake 102 can be aligned lengthwise along the axis 101. The stake 102 can have a tapered bottom end 104 integrated into the stake 102 (e.g., integrally formed with the stake 102 as a single piece) or attached to the bottom of the stake 102. The tapered bottom end 104 can be a pointed tip that helps guide the stake 102 into the soil or into a pre-formed bore for receiving the center of the stake 102. The tapered bottom end 104 can be injection molded for rigidity. As will be described below, the stake 102 can have a center portion having a generally cylindrical shape and a set of fins extending laterally away from the center portion (e.g., forming an x-shaped cross-section). The stake 102 can include a shell configured to contain and protect electronic components including electrodes for obtaining sensor measurements of the soil at one or more depths.

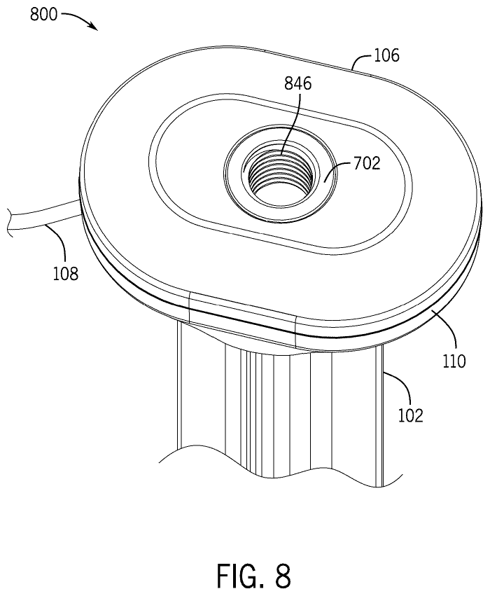

The dielectric sensor probe 100 can also include a cap 106 coupled to the stake 102. The cap 106 can alternatively be referred to as a top housing, box, or container. At least one side (e.g., a lower portion 126) of the cap 106 can include a visual indicator 110 (e.g., a visible horizontal line or marker) that acts as a reference for a user to visually identify whether the stake 102 is completely inserted into the ground. A top portion 124 of the cap 106 can include a rigid block 702 having a recess 846 or similar retention mechanism (see FIG. 8) configured to receive and retain a plug 112. As will be described below, the recess 846 can be used to attach tools or devices to the cap 106. The plug 112 can include a bolt, nut, cap, or cover to protect the recess 846 from being filled or clogged by fluids and debris (e.g., water, dirt, etc.).

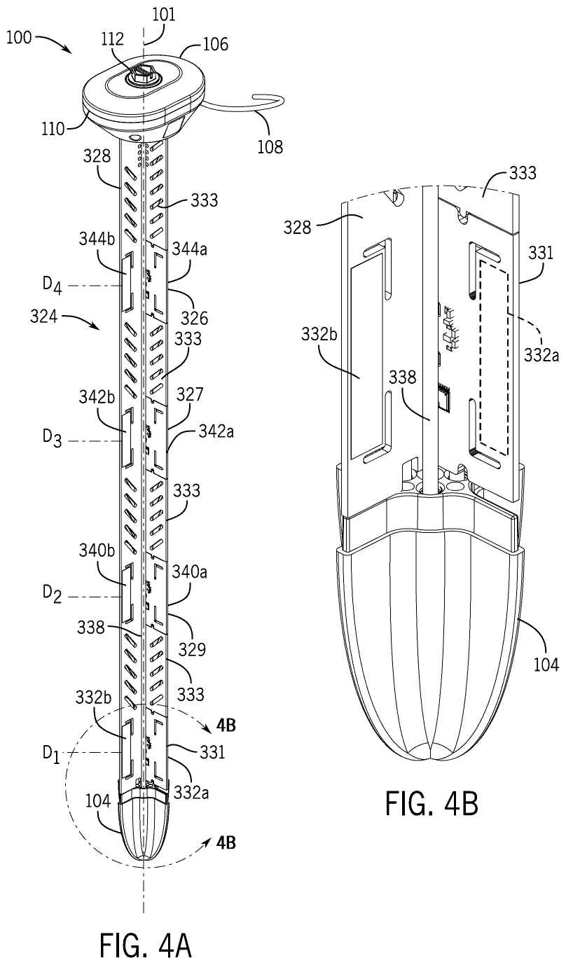

The stake 102 can include a set of reference indicators 114, 116, 118, 120. The reference indicators 114-120 can include lines, etchings, ridges, or other markings that correspond to specific distances from the visual indicator 110 of the cap 106, which may therefore correspond to specific depths within the soil when the stake 102 is fully inserted. Thus, in some examples, each reference line 114-120 corresponds to a depth at which one or more soil moisture level measurements are taken by a set of corresponding sensors (see also FIG. 4A-4B).

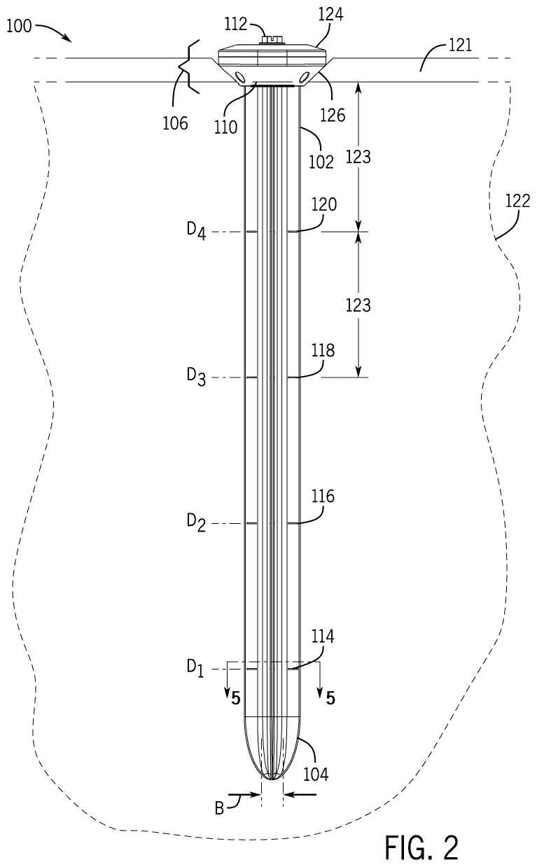

Any of the features, components, and/or parts, including the arrangements and configurations thereof shown in FIG. 1 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 1. Additional details of the dielectric sensor probe 100 in the ground are described below with respect to FIG. 2.

FIG. 2 illustrates a perspective view of the dielectric sensor probe 100 of FIG. 1 positioned within the ground, with the stake 102 inserted through loose debris/soil/plants 121 and into the packed soil 122 underneath. The tapered bottom end 104 of the stake 102 can be inserted into the ground 122 until the visual indicator 110 reaches the top of the packed soil 122. See also FIGS. 8-10 and their related descriptions herein. In some examples, a portion of the cap 106 can extend above the surface of the packed soil 122 (e.g., about 1 to about 4 inches) when the dielectric sensor probe 100 is positioned at the operational depth. In some other examples, the top surface the cap 106 can be inserted flush with the packed soil 122 surface when the dielectric sensor probe 100 is positioned at the operational depth. Insertion of the probe 100 with the visual indicator 110 at the surface of the packed soil 122 can ensure consistency between multiple different probes 100 installed within an area of measurement (e.g., a field of similar soil and crops).

Each of the reference lines 114-120 can be at a different distance from the visual indicator 110. In some examples, each of the reference lines 114-120 corresponds to a position of a distinct sensor along the stake 102, and thus a distinct measurement depth. For example, each reference line 114-120 can be spaced apart from the next reference line (or the visual indicator) by a sensor spacing distance 123 of about 10-20 centimeters so that the soil 122 can be measured at multiple significantly spaced-apart depths.

In some embodiments, a spacer ring can be added to the top end of stake 102 to adjust the spacing of the various measurement depths along the stake 102. For example, the spacer ring can wrap around the outer perimeter of the stake 102 and contact the bottom surface of the cap 106, and the spacer ring can have a predetermined longitudinal height, such as 5 centimeters. When the probe 100 is inserted into the ground, the spacer ring can contact the ground and space the cap 106 away from the ground by a distance equal to the predetermined longitudinal height. Accordingly, while a probe 100 without a spacer ring may have measurement depths at 15, 30, 45, and 60 centimeters below the ground surface, a probe with a 5 centimeter tall spacer ring may have measurement depths at 10, 25, 40, and 55 centimeters. A spacer ring with a different height can change the measurement depths similarly. By implementing a spacer ring, the measurement depths can be customized and optimized for various plant depths and soil properties by positioning the measurement depths higher than they would otherwise be. Furthermore, the spacer ring can help ensure that multiple probes are installed with their respective measurement depths substantially identical.

In some embodiments, the stake 102 can be installed at a non-perpendicular angle relative to the ground surface. By doing so, the longitudinal axis of the stake 102 can be angularly offset from a vertical direction. With the stake 102 extending at an offset angle relative to the vertical direction, the depth of each measurement depth (e.g., D₁-D₄ in FIG. 2) relative to the top surface of the packed soil 122 can be reduced as compared to when the stake 102 is vertically inserted. Thus, angular insertion of the stake 102 can help facilitate soil measurements at shallower depths and/or at more closely spaced depths (e.g., lower spacing distances 123) as measured from the solid ground surface.

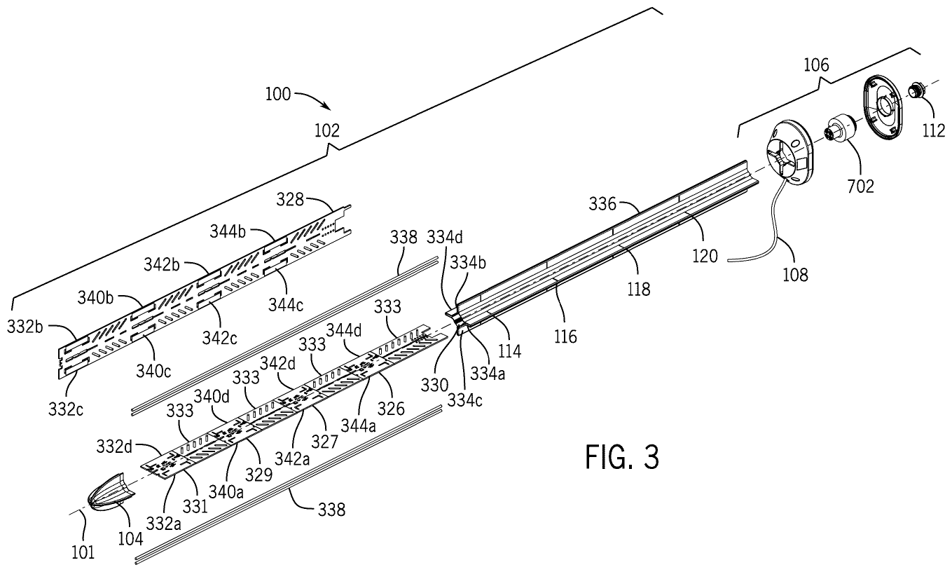

Any of the features, components, and/or parts, including the arrangements and configurations thereof shown in FIG. 2 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 2. Additional details of dielectric sensor probe components are described with reference to FIG. 3.

FIG. 3 illustrates an exploded view of the dielectric sensor probe 100. FIG. 4A shows the probe 100 with an outer shell 336 removed, and FIG. 4B shows a detail view of the bottom end of the probe 100 of FIG. 4A.

The dielectric sensor probe 100 can include an electronic component 324 positioned in the stake 102, including a set of circuit boards 326, 327, 328, 329, and 331. See FIGS. 3 and 4A. As described further below, the electronic component 324 can bear electrodes operable to sense dielectric permittivity of a quantity of material (such as soil, dirt, sand, or other porous material) between a pair of fins of the stake 102. A dielectric permittivity measurement can be used to determine a moisture level of the quantity of material.

The electronic component 324 can be positioned within the shell 336. The shell 336 can comprise a non-conductive material, such an extruded plastic. The shell 336 can include an opening 330 (i.e., a tunnel or through-hole) in which the electronic component 324 can be received. See FIG. 3. The electronic component 324 can be secured within the opening 330 by fasteners, adhesives, or similar devices. In some embodiments, the opening 330 can be filled with a hardened epoxy, resin, or similar substance binding the electronic component 324 to the shell 336 and holding the electronic component 324 in place within the shell 336.

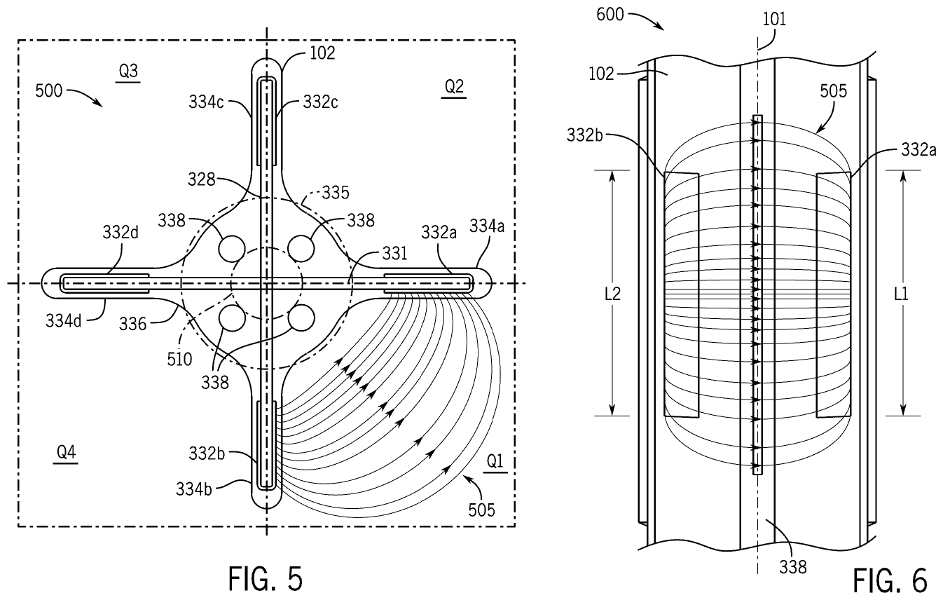

The stake 102 can include a first fin 334a and a second fin 334b extending along the longitudinal axis 101 of the shell 336. The first fin 334a and the second fin 334b extend radially away from the longitudinal axis 101 of the stake 102. The stake 102 or the shank body can have a cross-sectional profile including the first fin 334a and the second fin 334b extending radially from the central longitudinal axis 101. In some examples, the stake 102 can include at least two additional fins. The first fin 334a, the second fin 334, and the at least two additional fins can be arranged in an x-shape or cross-shape. The cross-sectional profile can include a central substantially cylindrical portion 335 (see the cross-section of FIG. 5), and the opening 330 can form an inner cavity extending through the shell 336, including the central substantially cylindrical portion 335.

In at least one example, the electronic component 324 includes at least a first circuit board 331 and a second circuit board 328. The first circuit board 331 and the second circuit board 328 can be arranged in an x-shape or a cross shape corresponding to the shape of the shell 336. At least a portion of the first circuit board 331 can be positioned within the stake 102 along the first fin 334a and at least a portion of the second circuit board 328 can be positioned within the stake 102 along the second fin 334b. For example, the first circuit board 331 and the second circuit board 328 can be arranged such that a first planar surface of the first circuit board 331 can be perpendicular to a second planar surface of the second circuit board 328. In some embodiments, the first circuit board 331 is electronically connected to the second circuit board 328 (i.e., capable of receiving and sending electrical signals through the second circuit board 328).

The electronic component 324 can include a set of circuit boards 326, 327, 329, 331 (e.g., daughter boards) electrically connected to a hub or larger circuit board 328 (e.g., mother board). The set of circuit boards 326, 327, 329, 331 can be spaced along the length of the larger circuit board 328 so that each smaller circuit board 326, 327, 329, 331 is positioned at a respective one of the depths D₁-D₄ along the stake 102, as shown in FIG. 4A. A set of spacers 333 can be positioned between each of the smaller circuit boards 326, 327, 329, 331 to ensure consistent spacing and a reliable connection of the circuit boards 326, 327, 329, 331 to the larger circuit board 328. In some embodiments, the spacers 333 and larger circuit board 328 may include diagonally-oriented through-holes, as shown in FIGS. 3 and 4A, to reduce weight and cost and to improve adhesive flow (e.g., epoxy flow) throughout the opening 330. The adhesive can thereby pass through the diagonal through-holes and harden, thereby reinforcing the positioning, rigidity, and strength of the spacers 333 and circuit board 328. In some embodiments, the smaller circuit boards 326, 327, 329, 331 and spacers 333 can be combined into a single integral piece, e.g., a single large circuit board similar in size and length to larger circuit board 328.

In at least one example, the electronic component 324 can include a first electrode 332a and a second electrode 332b positioned on two perpendicular circuit boards (e.g., 328 and 331), as shown in FIGS. 4A-4B. Additional electrodes 340 (including, for example, 340a-d), 342 (including, for example, 342a-d), 344 (including, for example, 344a-d) can be positioned at different positions along the length of the stake 102. Each of the electrodes 332, 340, 342, 344 can comprise a plate shape, such as a rectangular pad positioned on one or more side surfaces of their associated circuit board (e.g., 326, 327, 328, 331). The electronic component 324 can be in electronic communication with the first electrode 332a and the second electrode 332b. The first electrode 332a can have a first length measured along (e.g., parallel to) the longitudinal axis 101. The first electrode 332a can be positioned in the first fin 334a. The second electrode 332b can have a second length measured along the longitudinal axis 101. The second electrode 332b can be positioned in the second fin 334b with the first length overlapping with the second length (e.g., a short distance above and below D₁). Thus, the second electrode 332b can be angularly and laterally spaced apart from the first electrode 332a, and the first length and the second length can longitudinally overlap each other. In other words, the first electrode 332a and the second electrode 332b can be positioned at the same longitudinal distance from a reference point on the dielectric sensor probe 100 (e.g., the top of the cap 106, the visual indicator 110, bottom tip of the tapered bottom end 104, etc.)

The first electrode 332a can be positioned on the first circuit board 331, and thus the first electrode 332a is positioned in the first fin 334a. The second electrode 332b can be positioned on the second circuit board 328, and thus the second electrode 332b is positioned in the first fin 334b. The first electrode 332a and the second electrode 332b can be angularly offset from each other about the longitudinal axis 101, e.g., by an angular displacement of about 90 degrees. Thus, the electrodes 332a, 332b may be orthogonal to each other. As such, the electronic component 324 can be configured to measure the dielectric permittivity of a first quantity of material positioned between first electrode 332a and the second electrode 332b at a first ground depth D₁.

In at least one example, the electronic component 324 can further include a third electrode 332c. See FIG. 3. The third electrode 332c can have a third length measured along the longitudinal axis 101. The third electrode 332c can be spaced apart from the first electrode 332a, such as by being rotationally offset or angularly offset from the first electrode 332a by an angular displacement of about 90 degrees. In some examples, the third electrode 332c can also be spaced apart from the second electrode 332b. The first length and the third length can longitudinally overlap each other. The electronic component 324 can be operable to measure dielectric permittivity of a second quantity of material positioned between the first electrode 332a and the third electrode 332c. The second quantity of material can be offset from the first quantity of material by being positioned on an opposite side of the first electrode 332a and the first circuit board 331.

For example, the electronic component 324 can measure the dielectric permittivity between the first electrode 332a and the second electrode 332b corresponding to the electric permittivity of the material between those electrodes in a first section (e.g., in a first quadrant out of four quadrants of material surrounding the stake 102, wherein the first quadrant is positioned between two adjacent fins) at the first depth. See also FIG. 5 and its related descriptions herein. Similarly, the electronic component 324 can measure the dielectric permittivity between the first electrode 332a and the third electrode 332c corresponding to the electric permittivity of the material in a second section (e.g., in a second quadrant of material positioned between two different adjacent fins or between one of the fins of the first quadrant and another different fin) at the first depth. In other words, at least two electrodes can be coupled with the stake 102 or the shank body and can be operable to measure dielectric permittivity of the quantity of material positioned laterally adjacent to the at least two electrodes of the shank body.

The dielectric sensor probe 100 can include a set of rigid members 338 extending through an internal volume defined in the stake 102 and laterally surrounding the electronic component 324. The rigid members 338 are shown in FIGS. 3-7. In some examples, the internal volume can be filled with epoxy or resin to encompass at least the electronic component 324 and the rigid members 338. The rigid members 338 can be configured to transfer a load applied to a top end of the dielectric sensor probe 100 to a bottom tip of the dielectric sensor probe 100. As described below, a load may be applied longitudinally to the dielectric sensor probe 100, in particular, when installing or removing the dielectric sensor probe 100 from the ground.

Any of the features, components, and/or parts, including the arrangements and configurations thereof shown in FIG. 3, can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 3. Additional details of dielectric sensor probe components are described with reference to FIGS. 4A-4B.

The first electrode 332a can be positioned on the first circuit board 331 at a first longitudinal position corresponding a first measurement depth (e.g., D₁). The second electrode 332b can be positioned on the second circuit board 328 at the same first longitudinal position and angularly offset from the first electrode 332a (e.g., by 90 degrees about the longitudinal axis 101). As seen in FIG. 3, the third electrode 332c can be positioned on the second circuit board 328 at the first depth and angularly offset from the first electrode 332a (e.g., by 90 degrees in the opposite direction from the first electrode 332a as compared to the second electrode 332b). Similarly, a fourth electrode 332d can be positioned angularly offset from the second and third electrodes 332b, 332c (e.g., 90 degrees offset from each of the second and third electrodes 332b, 332c) on the first circuit board 331 at the same first depth. The first electrode 332a, the second electrode 332b, the third electrode 332c, and the fourth electrode 332d can provide a first set of soil moisture measurements at the first depth, wherein angularly adjacent pairs of the electrodes (e.g., electrodes 332a and 332b, electrodes 332a and 332c, electrodes 332b and 332d, or electrodes 332c and 332d) can each measure permittivity at the same depth D₁ for different quantities of material surrounding the stake 102. See also FIGS. 5-6. Electrodes 340a-340d, 342a-342d, and 344a-344d can provide similar measurements at respective depths D₂, D₃, and D₄.

Although the probe 100 of FIG. 4A has four sets of electrodes corresponding to four distinct measuring depths, in other examples, a dielectric sensor probe can have a fewer or more sets of electrodes (such as one, two, three, five, six, etc.) positioned at a different number of longitudinal positions and corresponding to a different number of measuring depths. For example, the number and depth of the discrete measuring depths at which electrodes are positioned can be optimized for a given crop or soil type, wherein crops with shallow root systems or shallow top soil/usable soil depth can be used with probes that have fewer or shallower measuring depths as compared to crops with deeper root systems and usable soil depths. In some examples, the sets of electrodes may be positioned at irregular distances from each other rather than being equally spaced apart, or sets of electrodes may have a different size (e.g., larger or smaller electrode plate dimensions) at different longitudinal positions. Additionally, although four fins and four corresponding electrodes are positioned at each depth in probe 100, thereby forming the x-shaped cross-sectional profile thereof, a different number of electrodes can be implemented at each depth, such as two, three, five, etc. Thus, in some examples, the cross-sectional profile of the stake 102 may comprise fins and internal circuit boards offset from each other by 120 degrees (for a three-finned stake), 72 degrees (for a five-finned stake), 60 degrees (for a six-finned stake), and so on. The number of fins may be optimized based on the soil type (e.g., since the number of fins is proportional to the friction that must be overcome during installation/removal of the stake 102), material used in the shell 336 (e.g., more durable material may be necessary to reinforce a larger number of fins), number of measurements required at each depth, etc. The number of fins may also affect sensor accuracy, wherein the angle between adjacent paired electrodes can beneficially be small enough to allow the electric field to pass directly/substantially linearly from one electrode to the other and large enough to allow a sufficient representative quantity of material to be sampled between the adjacent fins.

Any of the features, components, and/or parts, including the arrangements and configurations thereof shown in FIGS. 4A-4B can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 4A-4B. Additional details of electric fields to soil moisture measurements and are described with reference to FIG. 5.

FIG. 5 illustrates a simplified cross-sectional view diagram 500 of the stake 102 as taken through section lines 5-5 in FIG. 2. FIG. 6 shows a simplified side view diagram of the stake 102 at electrodes 332a, 332b with the shell 336 removed. The electrodes 332a, 332b can be operated to generate an electric field 505 passing primarily between their respective fins 334a, 334b and through a quantity of material positioned within one material zone (e.g., quadrant Q₁) next to the stake 102. Other pairings of electrodes 332 may be operated to generate a similar electric field through other material zones (e.g., in quadrants Q₂, Q₃, and Q₄). As discussed above, the stake 102 may also define an internal volume (e.g., within opening 330) containing at least the first circuit board 331, the second circuit board 328, the first electrode 332a, the second electrode 332b, the third electrode 332c, the fourth electrode 332d, and a set of rigid members 338.

In some examples, the cross-sectional shape of the stake 102 is at least partially rounded or circular, such as within the cylindrical portion 335 of the cross-section from which the fins 334a-d extend, as indicated in FIG. 5. The electric field 505 can be generated to form a portion of a ring shape (e.g., following a substantially rounded or circular path between the electrodes 332). The electric field 505 can extend between adjacent electrodes, such the first electrode 332a and the second electrode 332b positioned at the same measurement depth.

The permittivity of the material in a first material zone (e.g., Q₁) can be measured in view of the capacitance between the active electrodes (e.g., 332a, 332b) generating the electric field 505 through that zone. For example, the permittivity can be dependent upon or proportional to the capacitance. The capacitance, and therefore the dielectric permittivity, can also be directly dependent on water or moisture within the soil. Thus, soil having a higher water content may have a greater capacitance and dielectric permittivity than soil with a lower water content, so the permittivity measurements can indicate water content of the soil.

The electronic component 324 can be configured to perform a sequence of measurements at one or more depths. For example, the electronic component 324 can perform a first set of measurements at first soil depth D₁. In a first measurement, the electronic component 324 can determine a first dielectric permittivity of soil positioned between the first electrode 332a and the second electrode 332b (e.g., in Q₁). In a second measurement, the electronic component 324 can determine a second dielectric permittivity of soil positioned between the second electrode 332b and the fourth electrode 332d (e.g., in Q₄). In a third measurement, the electronic component 324 can determine a third dielectric permittivity of soil between the fourth electrode 332d and the third electrode 332c (e.g., in Q₃). In a fourth measurement, the electronic component 324 can determine a fourth dielectric permittivity of soil between the third electrode 332c and the first electrode 332a (e.g., in Q₂). Each of the first, second, third, and fourth measurements can be performed sequentially. While pairs of active electrodes generate the electric fields, the other pairs of inactive electrodes can have floating potential so as to not interfere with the electric fields generated by the active electrodes.

During the first measurement, the first electrode 332a and the second electrode 332b may be active and an electric field may be generated between them, while the third electrode 332c and the fourth electrode 332d may be floating (e.g., electrically disconnected from the first circuit board 326 and the second circuit board 328, respectively). Similarly, during the second measurement, the second electrode 332b and the fourth electrode 332d may be active, while the third electrode 332c and the first electrode 332a may be floating. The third and fourth measurements may be performed with similar floating electrodes. The activation or deactivation of any given electrode 332a-d from the corresponding circuit board may be achieved via switches, such as transistors, reed switches, piezoelectric switches, or other appropriate switch with a switching frequency of at least half of the predetermined measurement frequency.

In some examples, the sequence of measurements can be performed at a predetermined frequency by a clocking device. For example, the predetermined frequency can be 70 megahertz (MHz). In other examples the predetermined frequency can be greater than or less than 70 MHZ, and may be determined based on customer requirements, soil conditions, or the like.

In some examples, for each measurement, the electric field can be configured to be directed in the same angular direction (e.g., always clockwise or always counterclockwise). In other examples, the electric field can be configured to change directions (e.g., clockwise for a first period of time and counterclockwise for a second period of time). The direction of the electric field may be varied to detect variations or error in the permittivity measurements.

In some examples, the first, second, third, and fourth measurements can be compared or combined to determine a composite or estimated permittivity measurement for the overall material surrounding the stake 102. For example, multiple sensor measurements obtained at substantially the same depth and/or at substantially the same time of measurement can be collected and analyzed by a processor (e.g., of a computing device of the cap 106 or an external device) to determine a final estimated or combined measurement. Outlier measurements be caused by inconsistencies in the soil at the depth of the measurements, such as by rocks, voids, water pockets, sand, inconsistently packed soil, clay, or other irregularities in the ground affecting some of the electric fields at a given depth more than others. In one case, the processor can receive multiple (e.g., four) measurements and then implement electronic instructions including averaging the measurements (e.g., a normal mean or weighted average) or disregarding one or more measurements. Thus, outlier measurements or other expected errors can be mitigated to have little or no impact on the final combined measurement for that depth. In some embodiments, a set of measurements with the highest value of dielectric permittivity may be more heavily weighted than measurements with lower values of dielectric permittivity. In some examples, assigned weights and or discarding of measurements may be based on standard deviations from a mean/average value of the set of measurements, such as measurements outside a certain standard deviation value for the collected measurements being discarded while other measurements less than that standard deviation value being averaged or otherwise considered acceptable when calculating the permittivity.

It will be understood that the zones/quadrants of FIG. 5 may surround the stake 102 at each depth D₁-D₄, and therefore the electronic component 324 can separately perform similar sets of measurements for each set of electrodes 340a-340d, 342a-342d, and 344a-344d in order to obtain permittivity measurements at each depth.

Any of the features, components, and/or parts, including the arrangements and configurations thereof shown in FIG. 5 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 5.

As shown in the side view diagram of FIG. 6, the electric field 505 may be emitted between the electrodes 332a, 332b along their respective longitudinal lengths L1, L2. The first length L1 and the second length L2 can longitudinally overlap each other. In other words, the first electrode 332a and the second electrode 332b can be positioned at least partially at the same longitudinal position along the stake 102 and at least partially at the same measuring depth (e.g., D₁). The lengths L1 and L2 can beneficially be equal to each other and span the same longitudinal depth range in order to generate a consistent electric field and to minimize the distance the electrodes 332, thereby reducing signal error or noise.

In various examples, the longitudinal dimensions of the lengths L1 and L2 can be varied in order to vary the quantity of soil through which the electric field is generated. In addition, the strength of the magnetic field and the angle between the fins 334 and electrodes 332 can be varied in to change the range of the magnetic field and the quantity of soil sampled by the electrodes 332 along directions substantially parallel to the ground or perpendicular to the longitudinal axis 101. A narrower range of soil can provide a precise measurement at a specific depth while a greater width can provide a more general measurement. In some examples, to obtain a value at a given depth, the measurement across the width of soil depth can be averaged or otherwise processed (e.g., to obtain an average soil water content across an agricultural rooting zone).

Any of the features, components, and/or parts, including the arrangements and configurations thereof shown in FIG. 6 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 6.

As shown in FIGS. 3-7, the dielectric sensor probe 100 may include a set of rigid members 338 extending longitudinally through the internal volume of the stake 102. The rigid members 338 can be configured to transfer a load from the cap 106 of the probe 100 to the tapered bottom end 104. In some embodiments, as shown in FIGS. 3, 5, and 7, a set of multiple rigid members 338 (e.g., four) can be circumferentially spaced around the longitudinal axis 101 of the probe 100, such as by one rigid member 338 being positioned in each material zone/quadrant (e.g., Q₁-Q₄). In other examples, the set of rigid members can include one, two, three, five, six, etc. rigid members. The rigid members 338 can each be laterally or radially spaced away from the longitudinal axis 101 so as to provide a central space 510 through which the circuit boards of the electronic component 324 can connect and extend.

The set of rigid members 338 can extend between a rigid block 702 positioned in the cap 106 and the tapered bottom end 104. The set of rigid members 338 maybe configured to transfer a load applied to the rigid block 702 through the stake 102 and to the tapered bottom end 104. Although the electronic component 324, including the first circuit board 326 and the second circuit board 331 (and other circuit boards and spacers 333), may be structurally rigid, the set of rigid members 338 can provide additional rigidity and support to avoid damage to the electronic component while installing the probe 100, such as when a hammer blow is applied to the top end via the rigid block 702. For example, the set of rigid members 338 can provide longitudinal support to the stake 102 to prevent the stake 102 for breaking or bending when the dielectric sensor probe 100 is being installed. Additionally, the set of rigid members 338 can assist to pull the rigid block 702, stake 102, and tapered bottom end 104 from the ground when the dielectric sensor probe 100 is being removed.

In some examples, the rigid members 338 can comprise a rigid composite material such as fiberglass or carbon fiber/carbon-reinforced epoxy composites. In other examples, the rigid members can include metals (e.g., aluminum or steel) or polymers (e.g., polyvinyl chloride (PVC), polyethylene (such as high-density polyethylene (HDPE)), polycarbonate, etc.) or the like. In some examples, the rigid members can comprise electrically insulated materials, such as insulated metallic rods. In some examples, the rigid members can include internal lattice structures (e.g., via additive manufacturing) such as honeycomb, gyroid, etc. to provide additional structural rigidity.

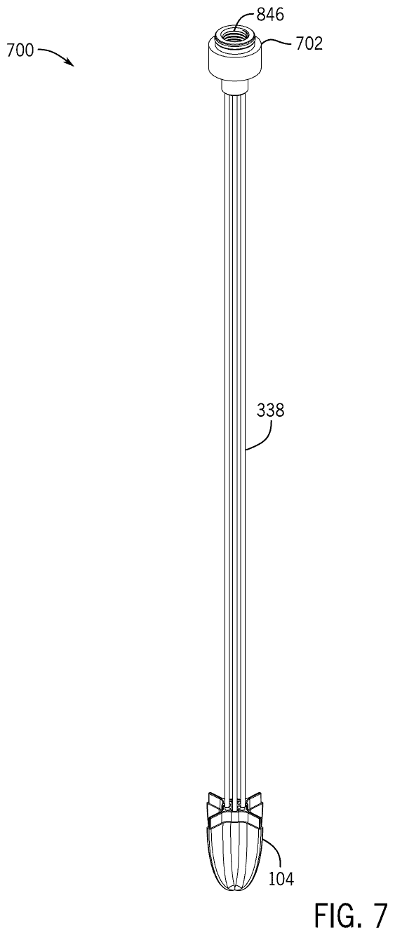

Any of the features, components, and/or parts, including the arrangements and configurations thereof shown in FIG. 7 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 7. Additional details of dielectric sensor probe components are described with reference to FIG. 8.

FIG. 8 illustrates a perspective view 800 of the top end of the dielectric sensor probe 100.

The cap 106 can contain various electronics, such as batteries, controllers, processors, electronic ports (e.g., for wire(s) or cable(s) 108), and the like. The cap 106 can cover the top end of the dielectric sensor probe 100 and protect internal components from water (e.g., from rain, sprinklers, runoff, etc.) and debris. For example, a bottom portion of the cap 106 can be configured to contact the ground. In some embodiments, the bottom surface of the cap 106 can be tapered to allow the bottom of the cap 106 to wedge into an opening at the top end of the bore that receives the stake 102, thereby limiting the amount of water and debris that can pass into the bore after installation, as illustrated in FIG. 2. The sides of the cap 106 can include one or more of the visual indicator 110. The visual indicator 110 can be a line, etching, ridge, or other marking that acts as a reference for a user to know the correct depth to place the dielectric sensor probe 100 into the ground.

A top end of the cap 106 can include a recess 846 formed in the top surface of the rigid block 702 and configured to receive the plug 112 (as illustrated in FIGS. 1-4A). In some examples, the recess 846 can include a threaded hole and the plug 112 can be a threaded bolt attachable to the threaded hole. In some examples, the recess 846 and the plug 112 can include one or more rods, pins (e.g., a cotter pin, R-clip, etc.) or other type of fastener to secure the plug 112 to the recess 846. In some embodiments, the recess 846 can be replaced by a protrusion (e.g., an extending portion, eyelet, hook, threaded shaft, or similar extending attachment body) configured to engage with an installation or removal tool. The recess or protrusion can be referred to as a retention mechanism of the cap 106 and/or the rigid block 702 of the cap 106.

The recess 846 or other retention mechanism can be used to attach tools or devices to the probe 100, such as to install or remove the dielectric sensor probe 100 from the ground. The plug 112 can be used to block and shield the recess 846 from water, dirt, etc. so that other tools can be engaged with the recess 846 as needed. The plug 112 can comprise a hexagonal/nut-shaped top portion configured to protrude from the cap 106 and to ease rotational attachment and removal of the plug 112 from the recess 846 (e.g., using a wrench).

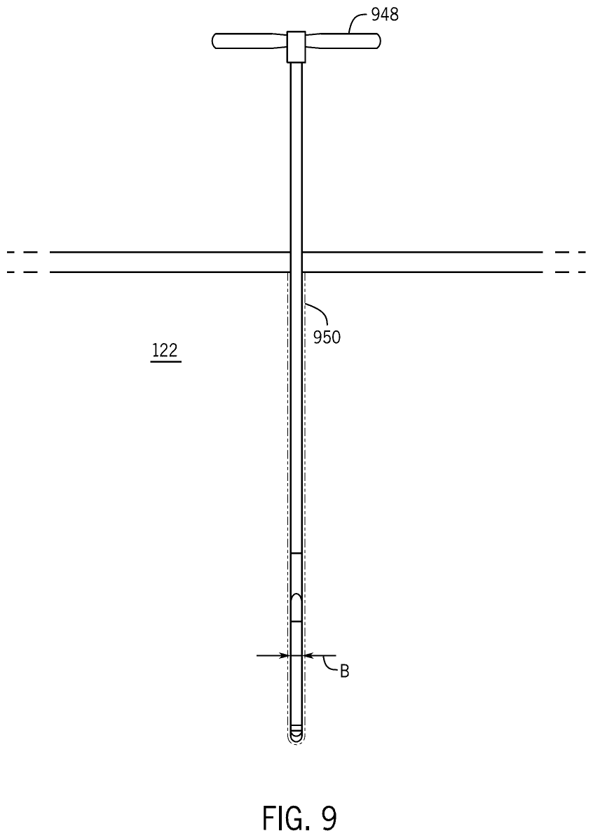

Any of the features, components, and/or parts, including the arrangements and configurations thereof shown in FIG. 8 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 8. Details of creating a bore into a porous medium to install a dielectric sensor probe are described with reference to FIG. 9.

FIGS. 9-13 illustrate an example process for installing and removing a sensor probe to or from soil. FIG. 9 illustrates an auger 948 for forming a bore 950 in the ground 122 before installing the dielectric sensor probe 100.

The auger 948 can be configured to form the bore 950 in the ground 122. The ground 122 can include soil or other porous medium. In some cases, the ground 122 can also include rocks, air pockets, or other non-homogeneities. The bore 950 may be configured to serve as a pilot hole into which the dielectric sensor probe 100 is inserted. The bore 950 can improve consistency of insertion direction of the probe 100 and reduce the driving force needed for inserting the probe 100, thereby minimizing the chances of pooling of water around the stake 102, which in turn can lead to inaccurate measurements of soil moisture.

The bore 950 can be formed with a diameter B approximately equal to a diameter of the central substantially cylindrical portion 335 of the stake 102 (e.g., the cross-sectional diameter of the stake 102 without including the fins 334a and 334b). See FIGS. 2 and 9. The bore 950 can be formed to a depth approximately equal to a distance between the visual indicator 110 of the cap 106 and the bottom point of the tapered bottom end 104 of the stake 102. Thus, the central substantially cylindrical portion 335 of the probe 100 can fit within the diameter B of the bore 950 while the fins 334 have an overall lateral width larger than the diameter B of the bore 950. In this manner, the tapered bottom end 104 of the dielectric sensor probe 100 can be inserted into the ground 122 at the location of the bore 950. The fins 334a and 334b can be inserted into the ground 122 external to the bore 950 diameter B.

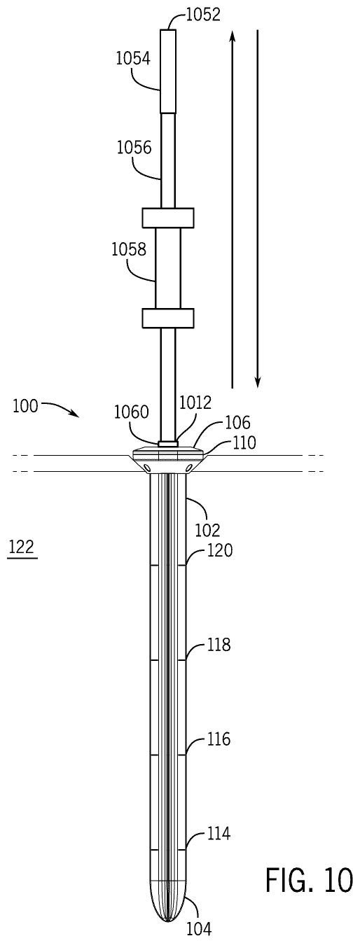

Any of the features, components, and/or parts, including the arrangements and configurations thereof shown in FIG. 9 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 9. Details of installing a dielectric sensor probe into a porous medium are described with reference to FIG. 10.

FIG. 10 illustrates a driving tool 1052 for inserting the dielectric sensor probe 100 into the ground 122. The driving tool 1052 can be coupled to a top end of the dielectric sensor probe 100, e.g., via being threaded into connection with the recess 846.

The driving tool 1052 can comprise a slide hammer or other hammering or impacting device. The driving tool 1052 can apply repeated impacts to the cap 106 or rigid block 702 (and therefore to the other parts of the rigid assembly, including rigid members 338 and tapered bottom end 104) to gradually drive the stake 102 into the ground 122. In some examples, the driving tool 1052 can include a handle 1054, a rod 1056, a drop weight 1058, and a connector 1060. The connector 1060 can be or can include a threaded end shaft for coupling the driving tool 1052 to the recess 846 of the cap 106. The handle 1054 can be held by a user who raises and drops the drop weight 1058. The force of the falling weight 1058 can be transferred to the probe 100.

The impacts may be applied to the top of the cap 106 or to the rigid block 702 to drive the stake 102 longitudinally into the bore 950. In some examples, a driving tool may not be needed, such as when the ground 122 is sufficiently soft that the dielectric sensor probe 100 can be driven into the ground 122 by hand. In some embodiments, a plate or shaft can be attached to the recess 846, and a sledgehammer or similar driving tool can be used to pound the probe 100 into the ground 122. Thus, various linear driving tools may be used for inserting the probe 100.

When the dielectric sensor probe 100 is inserted, the soil in the ground 122 is positioned between the electrodes 332a and 332b of the fins 334a and 334b. The fins 334 are wider than the diameter B of the bore 950 and therefore “cut” or “slice” into the soil beyond the bore 950 while being inserted. Thus, the fins 334 may laterally spread soil away from their outside surfaces while the probe 100 is pressed downward. The width of the fins 334 may be minimized to minimize the resistance of the probe 100 as it is being driven. Once the probe 100 is fully inserted into the ground, the ground material can be substantially flush against the outer sides of the fins 334 in a manner limiting or substantially preventing the formation of significant gaps into which water could otherwise collect, thereby improving sensor accuracy. Thus, the electric fields generated by the electrodes can pass through substantially only soil instead of through water-filled gaps, air gaps, or other installation artifacts that would reduce sensor accuracy and/or signal strength. The fins 334 and shell 336 may be rigid enough to substantially limit or prevent warping or bending of the fins 334 or shell 336 as they are driven into the ground 122. Thus, upon insertion, the fins 334 may be substantially straight and parallel to the longitudinal axis 101 of the probe 100.

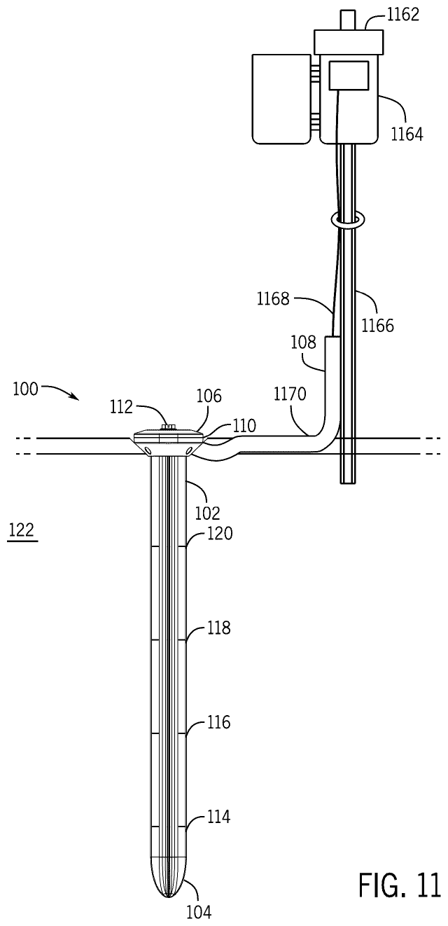

Any of the features, components, and/or parts, including the arrangements and configurations thereof shown in FIG. 10 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 10. Further details of operating a dielectric sensor probe are described with reference to FIG. 11.

FIG. 11 illustrates a data collection system 1162 for powering the dielectric sensor probe 100. The data collection system 1162 can include a junction box 1164, a post 1166, and a cable 1168. In some examples, at least a portion of the cable 1168 can be enclosed by a conduit 1170.

The post 1166 can secure the junction box 1164 into the ground 122. In some cases, the post 1166 can distance the junction box 1164 from the ground 122, such as to protect the junction box 1164 from damage due to water, dirt, equipment, etc.

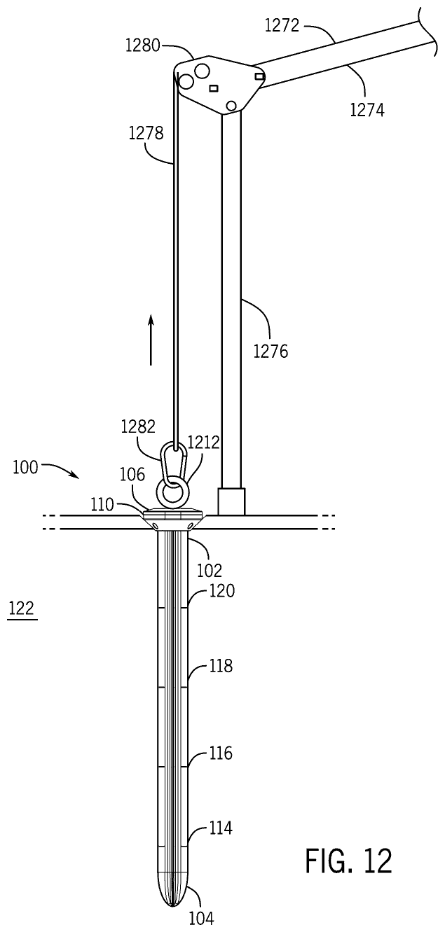

The cable 1168 can electrically and/or communicatively couple the junction box 1164 to the dielectric sensor probe 100. The cable 1168 can be the same as or similar to the cable 108. Alternatively, the cable 1168 can be electrically and/or communicatively coupled to the cable 108. The cable 1168 can provide power from the junction box 1164 to the dielectric sensor probe 100 in order to drive soil moisture measurements to measure dielectric permittivity of the soil. The cable 1168 can also send and/or receive information from the junction box 1164. In at least one example, the dielectric sensor probe 100 may send information regarding soil moisture levels. In at least one example, the dielectric sensor probe 100 may receive information regarding measurement parameters (switching frequency, on/off timers, etc.). Data collected by the sensor probe 100 can be transferred to the junction box 1164 to a data storage and/or collection device. The data collection system 1162 may therefore include a data storage device such as an electronic information storage medium (e.g., an electronic memory device) for retaining measurements. The data collection system 1162 can also comprise transmission equipment (e.g., antennae), processing equipment, and power generation and/or storage devices (e.g., solar panels, batteries, etc.). In some embodiments, the probe 100 contains an onboard energy storage device (e.g., a battery) for powering its sensor parts and transmitting data to the data collection system 1162. In some embodiments, signals from the probe 100 can be sent to the data collection system 1162 wirelessly (e.g., by a wireless communications medium such as Wi-Fi, BLUETOOTH®, radio frequency (RF) transmissions, infrared (IR) signals, or related methods).Any of the features, components, and/or parts, including the arrangements and configurations thereof shown in FIG. 11 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 11. Details of removing a dielectric sensor probe from a porous medium are described with reference to FIG. 12.

FIG. 12 illustrates a probe removal apparatus for removing the probe 100 from the ground 122. A removal lever 1272 may be used for removing the dielectric sensor probe 100 from the ground 122. The removal lever 1272 can include a handle 1274, a base 1276, a tension strap 1278 or cable, and a pivot 1280 or pulley system.

The tension strap 1278 can be mechanically connected to the cap 106 via one or more retention mechanisms. In at least one example, the retention mechanism 1212 can be an eye bolt engaging the recess 846. The tension strap 1278 can be coupled to the retention mechanism 1212 directly, via a hook, carabiner (e.g., 1282), or other mechanism.

The base 1276 can support the handle 1274. The user can apply a force to the handle 1274 to tension and/or wind the tensioning strap 1278 about the pivot 1280. The tensioning strap 1278 can apply a force to the cap 106 to remove the dielectric sensor probe 100 from the ground 122.

In some examples, the ground 122 can be sufficiently soft that the user can remove the dielectric sensor probe 100 from the ground by hand or by various other tools, such as a pry bar or simple lever engaging a retention mechanism (e.g., 1212 or a hook attached to the rigid block 702).

Any of the features, components, and/or parts, including the arrangements and configurations thereof shown in FIG. 12 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 12.

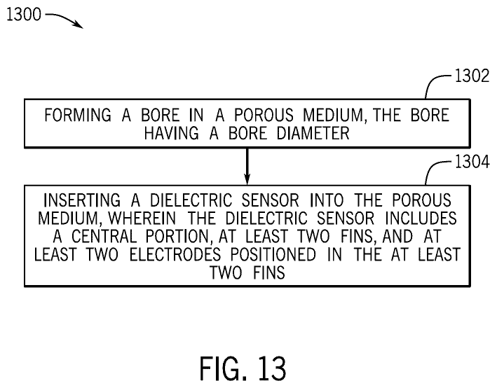

FIG. 13 is a flowchart illustrating a method 1300 of implementing a dielectric sensor in a porous medium.

At block 1302, the method 1300 includes forming a bore in a porous medium. The bore has a bore diameter, as discussed in connection with FIG. 9.

At block 1304, the method 1300 includes inserting a dielectric sensor into the porous medium, as discussed in connection with FIG. 10. The dielectric sensor includes a central portion, at least two fins (e.g., 334a, 334b), and at least two electrodes (e.g., 332a, 332b) positioned in the at least two fins, such as probe 100. Inserting the dielectric sensor into the porous medium includes inserting the central portion of the dielectric sensor into the bore and inserting the at least two fins into the porous medium external to the bore diameter. The porous medium is positioned between the at least two electrodes.

In a further example, the method 1300 can include coupling a driving tool (e.g., 1052) to a top end of the dielectric sensor and inserting the dielectric sensor into the porous medium via the driving tool.

In a further example, the method 1300 can include measuring permittivity of the porous medium using the at least two electrodes, e.g., as discussed in connection with FIGS. 5-6.

In a further example, the dielectric sensor includes at least one additional fin (e.g., 334c) and at least one additional electrode (e.g., 332c) positioned in the at least one additional fin.

In a further example, the method 1300 can include measuring permittivity of a first quantity of the porous medium using the at least two electrodes and measuring permittivity of a second quantity of the porous medium using one of the at least two electrodes and the at least one additional electrode.

While these systems and methods have been described with reference to exemplary embodiments, it will be understood by those skilled in the art that various changes may be made and equivalents can be substituted to adapt these teachings to other problems, materials, and technologies, without departing from the scope of the claims. Features, aspects, components or acts of one embodiment may be combined with features, aspects, components, or acts of other embodiments described herein. The disclosure is thus not limited to the particular examples that are disclosed, but encompasses all embodiments falling within the appended claims.

What is claimed is:

1. A dielectric sensor probe, comprising:
a stake having a longitudinal axis;
an electronic component positioned in the stake and including:
a first electrode having a first length measured along the longitudinal axis;
a second electrode having a second length measured along the longitudinal axis, the second electrode being spaced apart from the first electrode;
wherein the first length and the second length longitudinally overlap each other;
wherein the electronic component is operable to measure dielectric permittivity of a quantity of material positioned between the first electrode and the second electrode.

2. The dielectric sensor probe of claim 1, wherein the stake comprises a first fin and a second fin, wherein the first electrode is positioned in the first fin and the second electrode is positioned in the second fin.

3. The dielectric sensor probe of claim 1, wherein the first electrode and the second electrode are angularly offset from each other about the longitudinal axis.

4. The dielectric sensor probe of claim 1, wherein the electronic component comprises a first circuit board and a second circuit board arranged in an x-shape, the first electrode is positioned on the first circuit board, and the second electrode is positioned on the second circuit board.

5. The dielectric sensor probe of claim 1, wherein the stake comprises a shell, and the electronic component extends through the shell.

6. The dielectric sensor probe of claim 1, wherein the stake comprises a tapered bottom end.

7. The dielectric sensor probe of claim 1, wherein:
the electronic component comprises a third electrode having a third length measured along the longitudinal axis;
the third electrode is spaced apart from the first electrode;
the first length and the third length longitudinally overlap each other; and
the electronic component is operable to measure dielectric permittivity of a second quantity of material positioned between the first electrode and the third electrode.

8. The dielectric sensor probe of claim 1, wherein:
the electronic component comprises a third electrode having a third length measured along the longitudinal axis, the third electrode being spaced apart from the first electrode in a direction parallel to the longitudinal axis;
the electronic component comprises a fourth electrode having a fourth length measured along the longitudinal axis, the fourth electrode being longitudinally spaced apart from the second electrode;
the third length and the fourth length longitudinally overlap each other; and
the electronic component is operable to measure dielectric permittivity of a second quantity of material positioned between the third electrode and the fourth electrode.

9. The dielectric sensor probe of claim 1, further comprising a set of rigid members extending through an internal volume defined in the stake and laterally surrounding the electronic component, wherein the rigid members are configured to transfer a load applied to a top end of the dielectric sensor probe to a bottom tip of the dielectric sensor probe.

10. A soil sensor, comprising:
a shank body including a pointed tip, the shank body having a cross-sectional profile including at least two fins extending radially from a longitudinal axis of the shank body;
at least two electrodes coupled with the shank body and operable to measure dielectric permittivity of a quantity of material positioned laterally adjacent to the at least two fins of the shank body.

11. The soil sensor of claim 10, wherein the at least two electrodes comprise a first electrode positioned in a first fin of the at least two fins and a second electrode positioned in a second fin of the at least two fins.

12. The soil sensor of claim 10, wherein the at least two electrodes overlap a longitudinal position in the shank body.

13. The soil sensor of claim 10, wherein the cross-sectional profile includes at least two additional fins.

14. The soil sensor of claim 13, further comprising:
at least two additional electrodes coupled with the shank body in the at least two additional fins;
an electronic component in electronic communication with the at least two electrodes and the at least two additional electrodes and configured to sequentially measure dielectric permittivity of the quantity of material and at least one additional quantity of material laterally adjacent to the quantity of material.

15. The soil sensor of claim 10, wherein the cross-sectional profile includes a central substantially cylindrical portion defining an inner cavity.

16. A method of implementing a dielectric sensor in a porous medium, the method comprising:
forming a bore in a porous medium, the bore having a bore diameter;
inserting a dielectric sensor into the porous medium, wherein the dielectric sensor includes a central portion, at least two fins, and at least two electrodes positioned in the at least two fins;
wherein inserting the dielectric sensor into the porous medium includes inserting the central portion of the dielectric sensor into the bore and inserting the at least two fins into the porous medium external to the bore diameter, wherein the porous medium is positioned between the at least two electrodes.

17. The method of claim 16, further comprising coupling a driving tool to a top end of the dielectric sensor and inserting the dielectric sensor into the porous medium via the driving tool.

18. The method of claim 16, further comprising measuring permittivity of the porous medium using the at least two electrodes.

19. The method of claim 16, wherein the dielectric sensor comprises at least one additional fin and at least one additional electrode positioned in the at least one additional fin.

20. The method of claim 19, further comprising measuring permittivity of a first quantity of the porous medium using the at least two electrodes and measuring permittivity of a second quantity of the porous medium using one of the at least two electrodes and the at least one additional electrode.
